(19) United States
(12) Patent Application Publication
(10) Pub. No.: US 2002/0009734 A1
(43) Pub. Date: Jan. 24, 2002

(54) Mutations in human glutamate carboxypeptidase II gene impacting folate metabolism, and detection of affected individuals

(76) Inventors: Charles H. Halsted, Davis, CA (US); Angela M. Devlin, Walnut Creek, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/805,293

(22) Filed: Mar. 12, 2001

Related U.S. Application Data
(60) Provisional application No. 60/188,983, filed on Mar. 13, 2000.

(51) Int. Cl. C12Q 1/68; C12P 19/34

(52) U.S. Cl. 435/6; 435/91.2

(57) ABSTRACT

The invention provides methods for detecting mutations in the human GCPII gene which affect the ability of an individual to hydrolyze terminal glutamates from dietary folates. Such individuals are at increased risk for conditions associated with hyperhomocyteinemia, in particular, cardiovascular disease, colon cancer, and altered cognition in the elderly, including Alzheimer's disease. In addition, pregnant women with low folate status are at increased risk of bearing children with neural tube defects and congenital heart defects. Individuals with these mutations can be screened and treated with supplementation of their diet with folic acid.

### STATEMENT AS TO RIGHTS TO INVENTIONS MADE UNDER FEDERALLY SPONSORED RESEARCH AND DEVELOPMENT

This invention was made with government support under grants DK 45301 and DK 35747, awarded by the National Institute of Diabetes and Digestive and Kidney Diseases of the National Institutes of Health. The government has certain rights in the invention.

### CROSS-REFERENCES TO RELATED APPLICATIONS

This case claims priority from U.S. Provisional Patent Application No. 60/188,983, filed Mar. 13, 2000, the contents of which are incorporated by reference.

### BACKGROUND OF THE INVENTION

Folates are a family of vitamins that function in the transfer of methyl groups in the methionine cycle and DNA synthesis (Selhub, J. & Rosenberg, I. H. “Folic Acid” In Present Knowledge of Nutrition, Ziegler, E. E. & Filer, L. J. (eds.), ILSI Press, Washington, D.C., 206-219 (1996)). Low serum levels of folate, a condition termed “low folate status,” induce alterations in folate and methionine metabolism that result in hyperhomocysteinemia (Clarke, R. et al., Clin. Chem. 44:102-107 (1998)); Selhub, J. et al., JAMA 270:2693-2698 (1993)), which is associated with increased risk of cardiovascular disease (Boushey, C. J. et al., JAMA 274:1049-1057 (1995)), altered cognition (characterized by memory loss and loss of judgment) in the elderly (Joosten, E. et al., J. Gerontology 52:M76-79 (1997); Nilsson, K. et al., Eur. J Clin. Invest. 126:853-859 (1996); Clarke, R. et al., Arch. Neurol. 55:1449-1455 (1998); McCaddon, S. et al., Int. J. Geriatr. Psychiatry 13:235-239 (1998); Halsted, C. H. et al., J. Biol. Chem. 273:20417-20424 (1998)), and colon cancer (Giovannucci, E., et al., J. NCI 85:875-84 (1993)), and is common in mothers of children with neural tube defects (Mills, J. L. et al., Lancet 345:149-151 (1995)). Folate status is regulated in part by the intestinal absorption of dietary folylpoly-γ-glutamates, the predominant form of naturally occurring dietary folates. Folate absorption involves the sequential hydrolysis of terminal glutamate residues of folylpoly-γ-glutamates by folylpoly-γ-glutamate carboxypeptidase (FGCP), followed by membrane transport of the monoglutamyl folate derivatives by the reduced folate carrier (Halsted, C. H., “Intestinal absorption of dietary folates,” in Folic Acid Metabolism in Health and Disease, Picciano, M. F., et al., (eds.) Wiley-Liss, New York, pp. 23-45 (1990) (hereafter, “Halsted I”)). FGCP plays an important regulatory role in the availability of dietary folates, since folylpoly-γ-glutamates are absorbed at one half to one third less efficiency than monoglutamyl folates, according to studies in which physiologic amounts are perfused in the human jejunum (Halsted, C. H., supra; Halsted, C. H. et al., J. Clin. Invest. 59:933-940 (1977) (hereafter “Halsted II)).

Purified FGCP from pig and human small intestinal brush border membranes is a zinc-activated exopeptidase that hydrolyzes the terminal glutamate residues of folylpoly-γ-glutamates (Halsted II, supra; Chandler, C. J. et al., J. Biol. Chem. 261:928-933 (1986); Chandler, C. J. et al., Am. J. Physio. 260:G865-G872 (1991)). The nucleotide and predicted amino acid sequences of pig jejunal FGCP cDNA (Halsted, C. H. et al., J. Biol. Chem. 273:20417-20424 (1998) (hereafter, “Halsted III”)) are 88% and 92% identical to those of human prostate specific membrane antigen (PSMA) (Israeli, R. S. et al., Cancer Res 53:227-230 (1993)) and 83% identical to rat N-acetylated α-linked acidic dipeptidase (NAALADase) (Carter, R. E. et al., Proc. Natl. Acad. Sci. 95:3215-3220 (1998)), a brain enzyme that regulates glutamate neurotransmission by hydrolyzing the glutamate residue of N-acetyl-aspartyl-glutamate. The recent molecular characterization of human brain NAALADase cDNA confirmed its sequence identity to PSMA (Luthi-Carter, R. et al., J. Pharm. Exper. Therap. 286:1020-1025 (1998)). Pig jejunal brush border membranes and human prostate cancer PC3 cells transfected with the cDNA of pig FGCP exhibited both FGCP and NAALADase activity (Halsted III, supra) consistent with previous findings that PSMA is capable of hydrolysis of glutamate residues of folylpoly-γ-glutamates and N-acetyl-aspartyl-glutamate (Pinto, J. T. et al., Clin. Cancer Res. 2:1445-1451 (1996); Luthi-Carter, R. et al., Brain Res. 795:341-348 (1998)). This evidence supports the concept that FGCP, NAALADase and PSMA represent functionally distinct expressions of a single gene that encodes 750 amino acids and is collectively known as glutamate carboxypeptidase II (GCPII). Structural analysis of the predicted amino acid sequence of GCPII showed similarities with the peptidase family M28 and identified a single N-terminal membrane-spanning region and a catalytic region of 313 amino acids, which contains two co-catalytic zinc atoms (Rawlings, N. D. et al., Biochim. Biophys. Acta 1139:247-252 (1997)). This model was substantiated by the demonstration that NAALADase activity is reduced by site-directed mutagenesis of the predicted zinc binding sites of GCPII (Speno, H. S. et al., Mole. Pharm. 55:179-185 (1999)).

Several studies have described correlations between low folate status, hyperhomocysteinemia and dementia in aging Caucasian patients with Alzheimer's Disease (Joosten, E. et al., J. Gerontology 52:M76-79 (1997)); Nilsson, K. et al., Eur. J. Clin. Invest. 126:853-859 (1996)); Clarke, R. et al., Arch. Neurol. 55:1449-1455 (1998)); McCaddon, S. et al., Int. J. Geriatr. Psychiatry 13:235-239 (1998); Halsted III, supra. In a recent English study, significantly lower serum and red blood cell folate levels and higher serum homocysteine levels were found in a patient population of 164 patients with clinically diagnosed and histologically confirmed Alzheimer's disease compared with 108 of their age-matched controls, unrelated to the equal distribution of the C677T methylenetetrahydrofolate (MTHFR) mutation (Clarke, R. et al., Arch. Neurol. 55:1449-1455 (1998)) among the groups.

### SUMMARY OF THE INVENTION

The invention provides a method of screening an individual for increased risk of low folate status, said method comprising detecting a mutation in a human glutamate carboxypeptidase II (GCPII) gene in a biological sample from said individual, wherein detection of the mutation is indicative of decreased ability to hydrolyse a terminal glutamate residue of a folypoly-γ-glutamate, which decreased ability is associated with low folate status. The mutation can be a single nucleotide polymorphism (SNP). For example, the mutation can be a SNP causing a single amino acid substitution of H475Y.

The invention further provides methods of detecting such mutations. These methods include, for example, amplifying the GCPII gene, or a portion thereof containing the mutation, with a set of primers to provide an amplified product, sequencing the amplified product to obtain a sequence, and comparing the sequence of the amplified product with a known sequence of a wild-type GCPII gene, wherein a difference between the sequence of the amplified product and the sequence of the wild-type GCPII gene indicates the presence of a mutation. The amplification can be by any of a variety of techniques, such as PCR. In particular, mutations can be detected by amplifying exon 13 of the GCPII gene, subjecting said amplified exon 13 to digestion by restriction enzymes, separating the resulting restriction products to form a pattern of restriction fragment lengths, and comparing the pattern of restriction fragment lengths to a pattern of restriction fragment lengths formed by subjecting amplified exon 13 of a wild-type GCPII gene to the same restriction enzymes. The amplification can be by PCR. The separation of the restriction length fragments can be by gel electrophoresis, and the restriction enzyme can be AccI.

The mutations can also be detected by hybridization techniques. Conveniently, the sample nucleic acid is hybridized to a nucleic acid of known sequence, such as the wild-type GCPII gene or a portion thereof, or to a portion of the gene containing the mutation, under conditions sufficiently stringent that, if the reference nucleic acid is the wild-type sequence, failure of the sample to hybridize to the reference nucleic acid will indicate that it contains a mutation whereas hybridization will indicate it comprises the wild-type sequence. The converse will be true if the reference nucleic acid comprises a mutation. Either the sample nucleic acid or the reference nucleic acid can be immobilized on a support.

The invention further comprises a method of screening an individual for increased risk of low folate status comprising performing reverse transcriptase-PCR on mRNA from intestinal cells of the individual to amplify products of a GCPII gene, and determining the ratio of a variant product in which 93 bases of exon 18 are deleted to a normal product of the GCPII gene, wherein a ratio of the variant form to the normal form greater than 1:1.5, 1:2, or 1:2.5 indicates the individual is at increased risk of low folate status.

The invention further provides mutations in a GCPII gene which impairs the ability of a product of the gene to hydrolyse a conjugated folate to release folic acid compared to a product of a wild-type GCPII gene. Specifically, the mutations wherein the ability of a product of the gene to hydrolyse a conjugated folate is reduced by 20 percent or more compared to a product of a wild-type GCPII gene. Such mutations include the deletion of 93 bases resulting from a deletion of exon 18, and SNPs. In particular, one such mutation is a SNP causing an amino acid substitution of H475Y.

The invention further provides kits for detecting persons at increased risk for low folate status. Such kits include a container and appropriate primers for amplifying a GCPII gene or a portion thereof, and may further comprise an AccI restriction enzyme. Additionally, the kits may contain instructions for detecting mutations of the invention.

### BRIEF DESCRIPTION OF THE DRAWINGS

FIG. 1. Proposed exon map of human GCPII based on the genomic sequence of PSMA (O'Keefe, D. S. et al., Biochim. Biophys. Acta 1443:113-127 (1998)). The H475Y mutation is localized to exon 13, as shown by the “*” over the exon. The exon 18 deletion resulting from the splice variant is shown with hatched bars. The predicted regions coding for the various functional domains are based on the Rawlings and Barrett analysis (Rawlings, N. D. et al., Biochim. Biophys. Acta 1139:247-252 (1997)).

FIG. 2. Mutation analysis of human GCPII. A. An electropherogram showing the sequence from a portion of exon 13 revealed the C→T substitution in codon 475 that predicts replacement of histidine with tyrosine. B. An electropherogram showing the wild-type sequence. C. Restriction analysis of the H475Y mutation. Genomic DNA was amplified by PCR using primers flanking exon 13, producing a 244 bp fragment, followed by digestion with AccI and analysis on a 2.5% agarose gel. Digestion of mutated sequence produces 141 bp and 103 bp bands. Lane 1, ladder of DNA standards, lane 2, wild-type exon 13, lane 3 and 4, heterozygote samples.

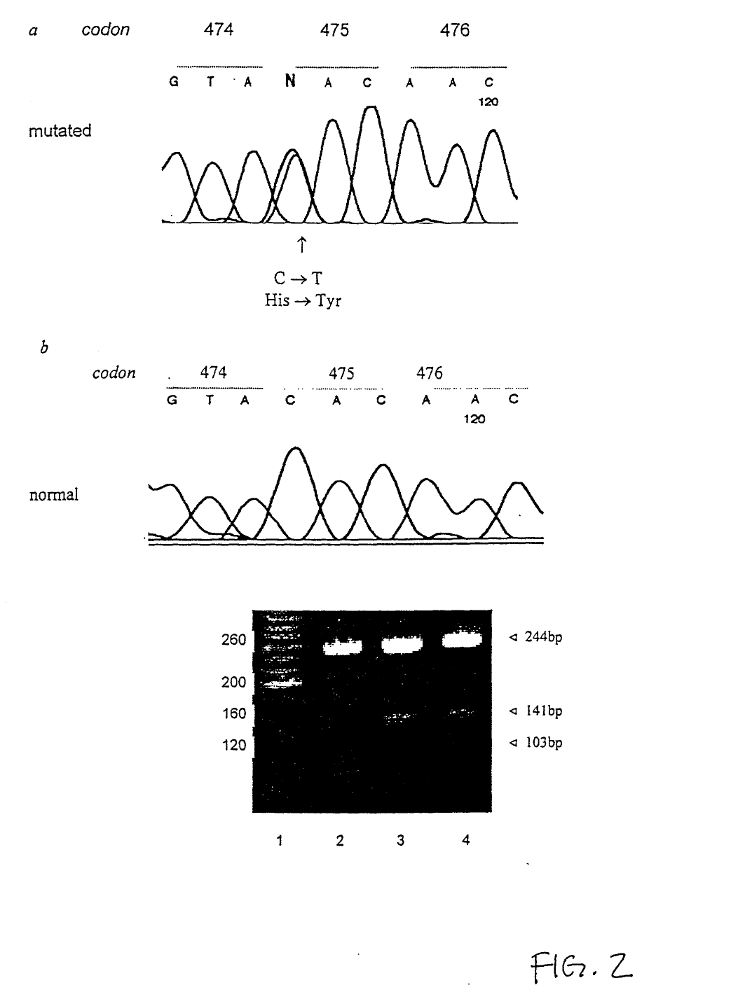

FIG. 3. FGCP activity in membranes of COS-7 cell transfectants. FGCP activity was determined in membranes from COS-7 cell transfectants using the substrate folyl-γ-glutamyl-γ-[¹⁴C]glutamate (Chandler, C. J. et al., J. Biol. Chem. 261:928-933 (1986)); Krumdieck, C. L. et al., Anal. Biochem. 35:123-129 (1970)). COS-7 cells were transfected with constructs of either GCPII cDNA (wild-type) or H475Y mutated GCPII cDNA (H475Y) in pTRACER-CMV2 (Invitrogen). Results show the means+SD of three experiments for each transfectant. There was no activity in mock transfected cells (not shown). FGCP activity was significantly lower (p<0.01) in membranes from H475Y mutant transfectants than membranes from wild-type GCPII transfectants.

FIG. 4. Cumulative frequency distributions of serum folate and homocysteine according to GCPII genotype. Legend: “+/−” denotes heterozygote of wild-type GCPII gene and GCPII gene with H475Y mutation; “−/−” denotes a homozygous GCPII gene.

### DETAILED DESCRIPTION

Introduction

We have now characterized the cDNA sequence of human intestinal GCPII and found complete identity to human PSMA (Israeli, R. S. et al., Cancer Res 53:227-230 (1993)) and NAALADase (Luthi-Carter, R. et al., J. Pharm. Exper. Therap. 286:1020-1025 (1998)), with some exceptions. These results confirm the concept that GCPII is expressed as three different proteins: intestinal FGCP, brain NAALADase and PSMA.

We have identified a splice variant of GCPII in human jejunum which lacks exon 18. The splice variant (“splice variant transcript”) lacks 93 nucleotides present in the normal expression product (a “wild-type GCPII transcript”). The intestinal FGCP enzyme translated from the splice variant (the “splice variant enzyme”) is inactive in hydrolyzing the conjugated folates which are the majority of folates present in the diet. Thus, the ratio of splice variant transcript to wild-type GCPII transcript is an important underlying factor governing the level of expression of intestinal FGCP activity and, potentially, brain NAALADase activity.

While the splice variant is present in some degree in all individuals, persons who have a higher ratio of splice variant transcripts to wild-type transcripts (for example, ratios of splice variants to wild-type transcripts of 1:1.5, 1:2, or 1:2.5) than do individuals with normal ratios, are less capable than the majority of persons at cleaving the terminal glutamates from dietary folates and are therefore less likely to be able to satisfy their needs for folate from dietary folates alone. Such persons are accordingly more likely to require supplementation of their diet with folic acid, which does not require hydrolysis before it can be efficiently absorbed in the intestines. In the absence of such supplementation, these persons are at increased risk for low folate status, at increased risk for hyperhomocysteinemia, and at increased risk for conditions associated with hyperhomocyteinemia, in particular, cardiovascular disease, colon cancer, and altered cognition in the elderly, including Alzheimer's disease. In addition, pregnant women with low folate status are at increased risk of bearing children with neural tube defects and congenital heart defects.

Individuals can be screened to determine if they are at increased risk of developing low folate status by determining the ratio of splice variant transcripts to wild-type transcripts, particularly in the intestinal tract. This is typically accomplished by taking an endoscopic biopsy of intestinal tissue, preparing mRNA from the sample, and determining the relative proportions of the mRNA transcripts by any of a variety of methods, such as quantitative PCR, RNase protection, real time PCR, and Northern blot analysis. Persons found to be at increased risk can be given folic acid supplementation and subjected to more frequent monitoring for low folate status and hyperhomocysteinemia and for the various conditions (such as cardiovascular disease, stroke, and colon cancer) associated with these conditions. Additionally, premenopausal women can be given folic acid supplementation above the normal recommendations of 400 μg/day, or 600 μg/day if pregnant, to reduce the risk that they will bear children with birth defects associated with folic acid deficiency.

We have further discovered a single nucleotide polymorphism, or “SNP,” in which a cytosine is replaced by a thymidine in codon 475 of GCPII. This results in the substitution of an amino acid at position 475 of the gene product from a histidine to a tyrosine (in shortened form, “H475Y”). Cells transfected with the mutant sequence proved to have only 56% of the ability to hydrolyse terminal glutamates from dietary folates. Studies further showed that the H475Y polymorphism in GCPII was significantly associated with lower serum folate and higher homocysteine levels in an aging Caucasian population. Thus, individuals positive for the H475Y GCPII allele are at increased risk for low folate status and consequent hyperhomocysteinemia. These individuals are therefore also at increased risk for cardiovascular disease, colon cancer, altered cognition, including Alzheimer's disease, and, in women, of bearing children with neural tube defects, congenital heart defects, or both.

Individuals can be screened to determine if they are at increased risk for these conditions by determining if they bear the H475Y polymorphism. Conveniently, such screening can be performed by taking a sample from an individual containing DNA (such as a blood sample) and amplifying the GCPII gene or a portion comprising codon 475. Since codon 475 is found in exon 13 of the gene, this can be conveniently performed with primers amplifying exon 13 or regions of the gene containing region 13.

Persons found to be at increased risk for low folate status because of a SNP, missense, or nonsense mutation can be subjected to more frequent monitoring for low folate status and hyperhomocysteinemia and for the various conditions (such as cardiovascular disease, stroke, and colon cancer) associated with these conditions. Additionally, premenopausal women can be given folic acid supplementation above the normal recommendation of 400 μg/day, or 600 μg/day for pregnant women, to reduce the risk that they will bear children with birth defects associated with folic acid deficiency. Males carrying these polymorphisms or mutations can be counseled that their children may have an increased risk of low folate status and be warned to provide them with folic acid supplementation.

Based on these two variants of the GCPII gene that impair or eliminate the ability of the FGCP enzyme to hydrolyze conjugated folates, it is anticipated that other mutations in the GCPII gene, such as nonsense and missense mutations and other gene defects, such as insertions and deletions, will likewise impair or even eliminate the ability of the resulting FGCP enzyme to hydrolyze conjugated folates and likewise be a marker for use in screening individuals at increased risk of low folate status and consequent hyperhomocysteinemia. Such mutations can easily be tested for their effect by the assays taught herein, such as transfecting cells which do not normally express the gene with the mutant gene and determining the activity of the enzyme expressed by the transfected cells. Persons found to have such mutations can be treated by supplementing their diet with folic acid to increase their serum folate levels, or “folate status,” to normal levels.

Definitions and Terms

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

Amino acid residues mutated from a known sequence are by convention designated by listing in standard single letter code the residue normally found at a designated position in the sequence, the position in the sequence of the residue mutated, and the residue substituted for the original residue. Thus, for example, “H475Y” indicates that a histidine residue normally found at position 475 of the relevant sequence has been replaced or substituted by a tyrosine.

As used herein, the “GCPII” gene refers to the glutamate carboxypeptidase II gene. The results reported here confirm that the GCPII gene is expressed in different tissues as the intestinal enzyme folypoly-γ-glutamate carboxypeptidase, as the prostate specific membrane antigen (“PMSA”), and as brain NAALALDase. The genomic sequence of the GCPII gene is the sequence previously published as encoding the PSMA gene by O'Keefe, D. S. et al., Biochim. Biophys. Acta 1443:113-127 (1998). The sequence is available in GenBank under Accession Number AF007544. As used herein, the term “wild-type GCPII gene” and references to the GCPII gene without further elaboration refers to the sequence set forth in GenBank under this accession number. The cDNA sequence of intestinal GCPII is in GenBank under accession number AF 176574 (this sequence was formerly under accession number M99487). The BLAST algorithm (Altschul, S. F. et al., Nucleic Acid Res. 25:3389-3402 (1997)) was used to compare the cDNA sequence to the genomic sequence and thereby derive the exons (which are present in the cDNA) discussed herein and the intron regions (which are the portions of the genomic DNA not present in the cDNA).

“FGCP” means folypoly-γ-glutamate carboxypeptidase, an intestinal brush border enzyme (designated EC 3.4.17.21) that is required to cleave the terminal glutamic acid residues from the conjugated folates found in the diet, releasing folic acid for intestinal absorption. FGCP is a product of the GCPII gene.

“Conjugated folates” means polyglutamyl folates. Cleavage of the terminal glutamic acid residues from conjugated folates releases a monoglutamic folate commonly referred to as “folic acid,” which is available for absorption by the intestines. Conjugated folates constitute over 95% of naturally-occurring folates in the diet (“dietary folates”) (Betterworth, C. E., Jr., et al., J. Clin. Invest 542:1929-1939 (1963)). “Low folate status” refers to an individual with folate levels which are associated with increased levels of homocysteine, and increased risk of colon cancer, cognitive defects such as Alzheimer's disease, and women at increased risk for bearing children with neural tube defects or congenital heart defects. A person in the lowest 20% of the range of normal blood serum folate is generally considered to have low folate status. Blood serum levels below 3 ng/ml (as measured by standard radioimmunoassays such as that supplied by BioRad (Hercules, Calif.) or 5 ng/ml (as measured by the standard microbiologic assay (Tamura, T., “Microbiological Scope of Folates,” in M. F. Picciano, et al., eds., Folic Acid Metabolism in Health and Disease, Wiley-Liss Co., New York (1990) at pp. 121-138, are generally considered to indicate that the individual has a low folate level.

“AccI” denotes a restriction enzyme derived from Acinetobacter calcoaceticus. It is commercially available from a number of sources, including New England Biolabs, Inc. (Beverly, Mass.) and Life Technologies, Inc. (Rockville, Md.).

The terms “stringent hybridization conditions” or “stringent conditions” refer to conditions under which a nucleic acid sequence will hybridize to its complement, but not to other sequences in any significant degree. Stringent conditions in the context of nucleic acid hybridizations are sequence dependent and are different under different environmental parameters. Longer sequences hybridize specifically at higher temperatures. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays,” Elsevier, N.Y., (1993) (the entirety of Tijssen is hereby incorporated by reference). Very stringent conditions are selected to be equal to the T_{M} point for a particular probe. Less stringent conditions, by contrast, are those in which a nucleic acid sequence will bind to imperfectly matched sequences. Stringency can be controlled by changing temperature, salt concentration, the presence of organic compounds, such as formamide or DMSO, or all of these. The effects of changing these parameters are well known in the art. The effect on Tₘ of changes in the concentration of formamide, for example, is reduced to the following equation: Tₘ=81.5+16.6 (log Na⁺)+0.41 (% G+C)−(600/oligo length)−0.63(% formamide). Reductions in Tm due to TMAC and the effects of changing salt concentrations are also well known. Changes in the temperature are generally a preferred means of controlling stringency for convenience, ease of control, and reversibility. Exemplary stringent conditions for detecting single nucleotide polymorphisms are well known in the art.

“Solid support” and “support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Methods for Amplification of the GCPII Gene or Portions Thereof

The GCPII gene or desired portions thereof, such as exon 13, can be amplified by cloning or by other methods, such as the polymerase chain reaction (PCR), the ligase chain reaction (LCR), the transcription-based amplification system (TAS), the self-sustained sequence replication system (SSR). These and other amplification methodologies are well known to persons of skill. Examples of these techniques and instructions sufficient to direct persons of skill through cloning exercises are found in Berger and Kimmel, Guide to Molecular Cloning Techniques, Methods in Enzymology Vol. 152, Academic Press, Inc., San Diego, Calif. (1987) (hereinafter, “Berger”); Sambrook et al., Molecular Cloning—A Laboratory Manual (2nd ed.) Vol. 1-3, Cold Spring Harbor Laboratory, Cold Spring Harbor Press, NY (1989), (“Sambrook et al. ”); Current Protocols in Molecular Biology, F. M. Ausubel et al., eds., Current Protocols, Greene Publishing Associates, Inc./John Wiley & Sons, Inc., (1994 Supplement) (“Ausubel”); Cashion et al., U.S. Pat. No. 5,017,478; and Carr, European Patent No. 0 246 864. Examples of techniques sufficient to direct persons of skill through other in vitro amplification methods are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (“Innis”); Arnheim & Levinson (Oct. 1, 1990) C&EN 36-47; J. NIH Res., 3: 81-94 (1991); Kwoh et al., Proc. Natl. Acad. Sci. USA 86: 1173 (1989); Guatelli et al., Proc. Natl. Acad. Sci. USA 87, 1874 (1990); Lomell et al. J. Clin. Chem., 35: 1826 (1989); Landegren et al., Science, 241: 1077-1080 (1988); Van Brunt, Biotechnology, 8: 291-294 (1990); Wu and Wallace, Gene, 4: 560 (1989); and Barringer et al., Gene, 89: 117 (1990).

In one preferred embodiment, the human GCPII gene can be isolated by routine cloning methods. The cDNA sequence provided in GenBank under Accession Number M99487 can be used to provide probes that specifically hybridize to the GCPII gene in a genomic DNA sample (Southern blot), or to the GCPII mRNA, in a total RNA sample (e.g., in a Northern blot), or to cDNA reverse-transcribed from RNA (in a Southern blot)). Once the target GCPII nucleic acid is identified (e.g., in a Southern blot), it can be isolated according to standard methods known to those of skill in the art (see, e.g., Sambrook et al., supra; Berger, supra, or Ausubel, supra). One method of cloning the the GCPII gene is provided in the Examples.

In another preferred embodiment, the human GCPII cDNA can be isolated by amplification methods such as polymerase chain reaction (PCR). Suitable primers for the amplification of the various exons of the gene are set forth in the Examples.

Labeling of Nucleic Acid Probes

Where the GCPII cDNA or its subsequences are to be used as nucleic acid probes, it is often desirable to label the sequences with detectable labels. The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In another preferred embodiment, transcription amplification using a labeled nucleotide (e.g. fluorescein-labeled UTP and/or CTP) incorporates a label into the transcribed nucleic acids.

Alternatively, a label may be added directly to an original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled DNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²p), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

Detecting Mutations in the GCPII Gene

There are a number of methods known in the art for detecting mutations in a given gene. Mutations in the gene can be found directly by amplifying the gene in a biological sample, such as white blood cells drawn from an individual being screened for a mutation in the gene, and comparing the sequence of the individual's GCPII gene to that of the wild-type GCPII gene sequence. Conveniently, the gene can be sequenced by pyrosequencing, as taught in, e.g., Ronaghi et al. Science 281:363-365 (1998) and Ronaghi et al. Anal. Biochem. 242:84-89 (1996). In this technique, a sequencing primer is hybridized to the gene and incubated with DNA polymerase, ATP sulfurylase, firefly luciferase, and a nucleotide-degrading enzyme. A deoxynucleotide is then added. If the deoxynucleotide is complementary to the base in the template strand, it will be incorporated, and the incorporation will be accompanied by release of pyrophoshate equal in molarity to that of the incorporated deoxynucleotide. Unicorporated deoxynucleotides are degraded by the degrading enzyme (which can be, for example, a pyrase). If the deoxynucleotide is incorporated, pyrophosphate is released and converted to ATP by ATP sulfurylase and the concentration of ATP is then signaled by the luciferase, permitting real-time detection of the incorporation of the deoxynucleotide and, hence, of the sequence of the strand of DNA of the template.

Alternatively, one can separately digest samples of the individual's GCPII gene and of the wild-type GCPII gene with one or more restriction enzymes, separate the resulting fragments by electrophoretic techniques routine in the art (such as those taught in Ausubel), and compare the pattern of the fragments. A difference in the pattern of the fragments of the sample from the individual compared to that of the wild-type GCPII gene indicates a mutation is present in the individual being screened.

The mutation of histidine to tyrosine at position 475 of the GCPII gene, for example, results in the creation of an AccI restriction site. When amplified exon 13 of wild-type GCPII is treated with AccI, it yields a 244 base product. When amplified exon 13 of GCPII having the H475Y mutation is treated with AccI, it yields products of 141 and 103 bases. Accordingly, the presence of the mutation can be readily detected.

The nature of the mutation can be determined by, for example, sequencing the gene isolated from the individual. If the specific mutation found is not one already identified as resulting in impaired ability of the enzyme expressed from the gene, the mutation can be tested by any of a variety of standard methods to determine the effect of the mutation. For example, the gene can be transfected into an appropriate eukaryotic cell, the gene expressed, and the activity of the resulting enzyme compared for activity against the enzyme expressed by identical cells transfected with a wild-type GCPII gene. Standard assays for determining the activity of the FGCP enzyme are known in the art. An exemplary assay for transfecting cells and determining the activity of the FGCP enzyme is set forth in the Examples herein.

Another method known in the art is CFLP-cleavase fragment length polymorphism. This method involves amplifying the gene of interest, here GCPII, followed by digestion with cleavase I, which cuts the DNA at sites dependent on secondary structure. Results are resolved on agarose gels and different patterns of cleavage digestion products are obtained for wild-type and mutant samples.

A further method known in the art is temperature modulation heteroduplex chromatography (TMHC). This method was used in the original screening for the GCPII gene, as discussed in Examples, below. The method involves amplification of the gene of interest, here the GCPII gene, followed by denaturing of the PCR products and then slowly cooling, to a predetermined temperature based on the composition of the sample. While cooling the PCR products renature forming hetero and homoduplexes which are resolved from one another using TMHC. Conveniently, the resolution is performed using a WAVE® DNA fragment analysis system (Transgenomic, Inc., San Jose, Calif.).

In another set of embodiments, mutations in the GCPII gene are detected by hybridizing the gene or portions thereof from a biological sample, such as from an individual being screened, against a reference nucleic acid, such as the wild-type GCPII gene or one or more GCPII genes with a known mutation, such as the deletion of exon 18, or a H475Y substitution (for ease of discussion herein, the reference nucleic acids will be termed “probes” and the sample being screened the “nucleic acid of interest”).

The hybridizations can be performed while either the probes or the nucleic acids of interest are attached to solid supports, or while they are in a fluid environment.

In one set of embodiments, the hybridizations are performed on a solid support. For example, the nucleic acids of interest (or “samples”) can be spotted onto a surface. Conveniently, the spots are placed in an ordered pattern, or array, and the placement of where the nucleic acids are spotted on the array is recorded to facilitate later correlation of results. The probes are then hybridized to the array. Conversely, the probes can be spotted onto the surface to form an array and the samples hybridized to that array.

The composition of the solid support can be anything to which nucleic acids can be attached. It is preferred if the attachment is covalent. The material for the support for use in any particular instance should be chosen so as not to interfere with the labeling system to be used for the probes or the nucleic acids. For example, if the nucleic acids are labeled with fluorescent labels, the material chosen for the support should not be one which fluoresces at wavelengths which would interfere with reading the fluorescence of the labels.

Preferably, the support is of a material to which the samples and probes bind or one which is substantially non-porous to them, so that the oligonucleotides remain accessible (i.e., to the probes or the samples) at the surface of the support. Membranes porous to the nucleic acids may be used so long as the membrane can bind sufficient amounts of nucleic acid to permit the hybridization procedures to proceed. Suitable materials should have chemistries compatible with oligonucleotide attachment and hybridization, as well as the intended label, and include, but are not limited to, resins, polysaccharides, silica or silica-based materials, glass and functionalized glass, modified silicon, carbon, metals, nylon, natural and synthetic fibers, such as wool and cotton, and polymers.

In some embodiments, the solid support has reactive groups such as carboxy- amino- or hydroxy groups to facilitate attachment of the oligonucleotides (that is, the samples or the probes). Plastics may be used if modified to accept attachment of nucleic acids or oligonucleotides (since plastic usually has innate fluoresence, the use of non-fluorescent labels is preferred for use with plastic substrates. If plastic materials are used with fluorescent labels, appropriate adjustments should be made to procedures or equipment, such as the use of color filters, to reduce any interference in detecting results due to the fluorescence of the substrate). Polymers may include, e.g., polystyrene, polyethylene glycol tetraphtalate, polyvinyl acetate, polyvinyl chloride, polyvinyl pyrrolidone, buty rubber, and polycarbonate. The surface can be in the form of a bead. Means of attaching oligonucleotides to such supports are well known in the art, and are set forth, for example, in U.S. Pat. Nos. 4,973,493 and 4,569,774 and PCT International Publications WO 98/26098 and WO 97/46313. See also, Pon et al., Biotechniques 6:768-775 (1988); Damba, et al., Nuc. Acids Res. 18:3813-3821 (1990).

Alternatively, the samples can be placed in separate wells or chambers and hybridized in their respective well or chambers. The art has developed robotic equipment permitting the automated delivery of reagents to separate reaction chambers, including “chip” and microfluidic techniques, which allow the amount of the reagents used per reaction to be sharply reduced. Chip and microfluidic techniques are taught in, for example, U.S. Pat. No. 5,800,690, Orchid, “Running on Parallel Lines” New Scientist, Oct. 25, 1997, McCormick, et al., Anal. Chem. 69:2626-30 (1997), and Turgeon, “The Lab of the Future on CD-ROM?” Medical Laboratory Management Report. Dec. 1997, p.1. Automated hybridizations on chips or in a microfluidic environment are contemplated methods of practicing the invention.

Although microfluidic environments are one embodiment of the invention, they are not the only defined spaces suitable for performing hybridizations in a fluid environment. Other such spaces include standard laboratory equipment, such as the wells of microtiter plates, Petri dishes, centrifuge tubes, or the like can be used.

The above techniques can be used to locate SNPs, missense, and nonsense mutations in the gene of interest. Given the size of the GCPII gene (˜100,000 bases), it is convenient to amplify the gene in sections. For example, the gene can be amplified exon by exon, by intron, or at the exon-intron borders. Mutations in the introns or at the exon/intron borders, for example, may result in improper splicing. Specific primers for amplifying the exons of GCPII are set forth in the Examples. A multitude of other primers could, however, be generated from the sequence of the genomic DNA, following the teachings of Ausubel, Innis, and other standard references. Additionally, methods directed to detection of mRNA transcripts, such as reverse-transcriptase PCR (see, e.g., Ausubel, supra), can be used to detect splice variants. An example of such a technique is detailed in the Examples, below.

In addition to looking at the gene itself or at the mRNA transcribed from the gene, one can detect mutations by detecting mutated forms of the protein encoded by the gene. A mutation that results in a truncated protein or one with a conformation other than that of the normal enzyme can be expected to have epitopes which are not present on the normal enzyme. These mutated forms of the enzyme can be used to raise antibodies. Methods of producing polyclonal and monoclonal antibodies are known to those of skill in the art. See, e.g., Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif.; Goding (1986) Monoclonal Antibodies: Principles and Practice (2d ed.) Academic Press, New York, N.Y.; Birch and Lennox, Monoclonal Antibodies: Principles and Applications, Wiley-Liss, New York, N.Y. (1995).

Antibodies so raised are generally tested by being absorbed against the normal enzyme (conveniently, the enzyme is immobilized on a column by standard methods and the antibodies are run over the column) to remove those which cross react with the form of the enzyme expressed by the normal GCPII gene. If the antibodies remaining after being absorbed against the normal form of the enzyme bind to the form of the enzyme found in the individual being screened, it indicates that the conformation of the enzyme is abnormal and indicates the presence of a mutation in the gene.

### EXAMPLES

### Example 1

Characterization of Human Intestinal GCPII.

To characterize GCPII from human intestine and to confirm its identity with human PSMA/ NAALADase cDNA, two commercial human intestinal cDNA libraries were screened with an 852 bp PCR fragment of the ORF of pig jejunal FGCP cDNA and two two overlapping clones identified. Clone pFGCP72 consisted of 953 bp in the 5′ region encompassing 710 bp of the open reading frame and 43 bp of the 5′ untranslated region. Clone pFGCP52 consisted of 1534 bp spanning the 3′ region encompassing 1407 bp of the open reading frame and 127 bp of the 3′ untranslated region including the polyA tail. Sequence analysis showed that these two clones were 100%, 88%, and 83% homologous to corresponding regions in human PSMA cDNA (Israeli, R. S. et al., Cancer Res 53:227-230 (1993)), pig FGCP CDNA (Halsted, C. H. et al., J. Biol. Chem. 273:20417-20424 (1998)) and rat GCPII cDNA (Carter, R. E. et al., Proc. Natl. Acad. Sci. 95:3215-3220 (1998)), respectively, with the exception of a 93 bp deletion in the 3′ end of clone pFGCP52. Further analysis using the genomic sequence of PSMA (O'Keefe, D. S. et al., Biochim. Biophys. Acta 1443:113-127 (1998)) identified the 93 bp deletion as exon 18. Subsequently, both the splice variant and full- length transcripts were identified by RT-PCR of normal human jejunal mucosa obtained at elective gastric bypass surgery in an obese patient. The splice variant is believed to be universal in human small intestine since it was consistently identified by RT-PCR but at much lower levels than the full-length transcript in duodenal biopsy samples (Devlin, A. M. et al., Gastroenterology 116:A874 (1999)). Analysis using RACE recovered an additional 83 bp of the 5′UTR of intestinal GCPII that placed the transcriptional start site at −138 relative to the translation initiation codon.

A commercial multiple human tissue Northern Blot (Clontech) was probed with clone pFGCP52 to determine the tissue distribution of GCPII mRNA. A predominant 2.8 kb band was identified in prostate, small intestine, brain, kidney, liver and spleen, listed in order of decreasing intensity. A less prominent 1.5 kb band was identified in liver and kidney and a 1.0 kb band was found in placenta.

### Example 2

Mutation Analysis of GCPII

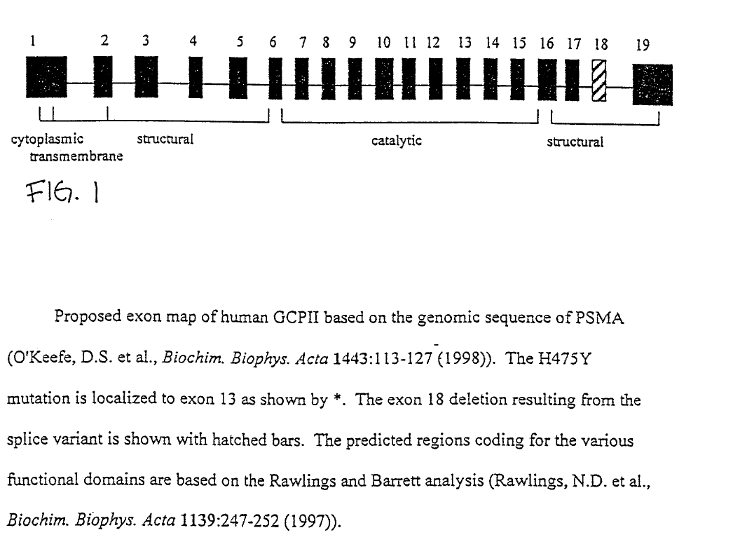

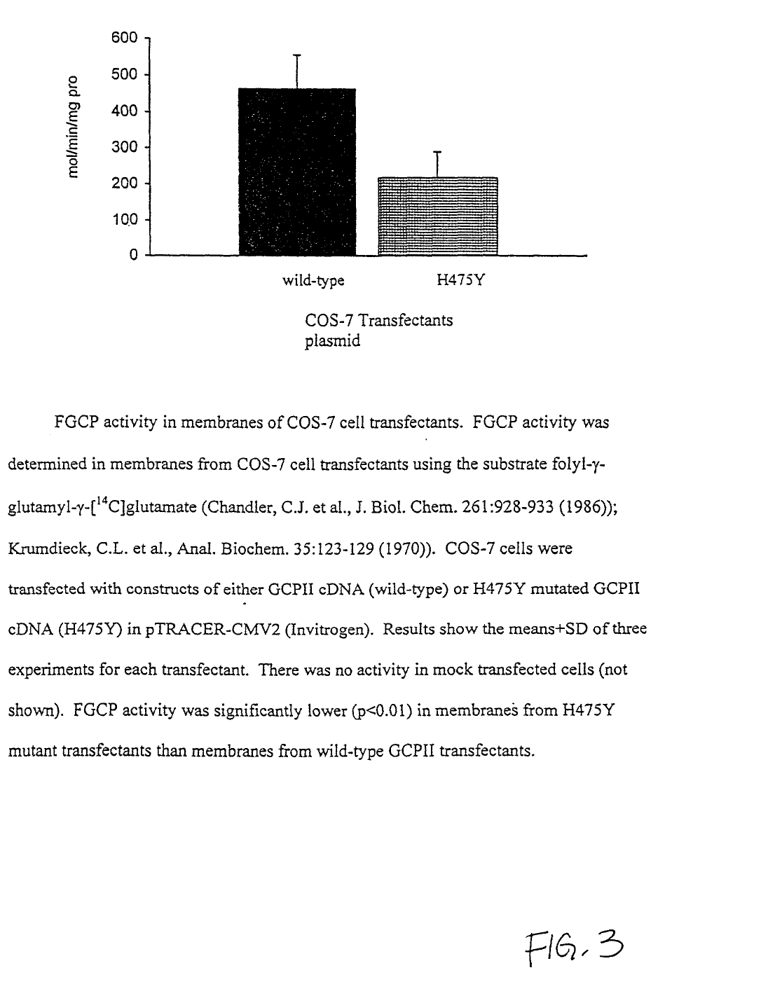

To identify a potentially significant mutation in GCPII that could contribute to a reduction in folate levels with consequent hyperhomocysteinemia, a subset of 181 DNA samples obtained retrospectively from a previous study of aging Caucasian subjects residing in Oxford, England (Clarke, R. et al., Arch. Neurol. 55:1449-1455 (1998)) were screened for GCPII polymorphisms. PCR primers were designed based on the human PSMA genomic sequence (O'Keefe, D. S. et al., Biochim. Biophys. Acta 1443:113-127 (1998)) to amplify the exon/intron borders and exons of human GCPII. (FIG. 1). Temperature modulated heteroduplex chromatography (Kuklin, A. et al., Genetic Testing 1:201-206 (1997/1998)) identified a polymorphism in exon 13. This finding was confirmed by sequencing the PCR products, identifying a missense mutation characterized by a C→T single base substitution in codon 475, predicted to replace a histidine with tyrosine (FIGS. 3A, B). The C→T substitution in codon 475 creates an AccI site (FIG. 3C). PCR followed by digestion with AccI was used to screen additional DNA samples for the H475Y GCPII mutation, followed by sequence confirmation of positive samples. This process yielded 18 heterozygous mutants and no homozygous mutants. Thus, the overall allele frequency of the H475Y mutation in the total population of 181 DNA samples was determined to be 0.05 with the genotype frequency being 10% heterozygote and 90% homozygote wild-type.

We studied in vitro the functional significance of the H475Y GCPII mutation in relation to FGCP activity. Mammalian COS-7 cells predetermined to lack endogenous FGCP activity were transfected with plasmids containing wild-type GCPII cDNA or H475Y mutant GCPII cDNA. Compared to FGCP activity in wild-type GCPII COS-7 transfectants, the H475Y GCPII mutation resulted in a significant 56% reduction (p<0.05) in FGCP activity (FIG. 3).

### Example 3

Relationship of the H475Y GCPII Mutation to Folate Status and Hyperhomocysteinemia.

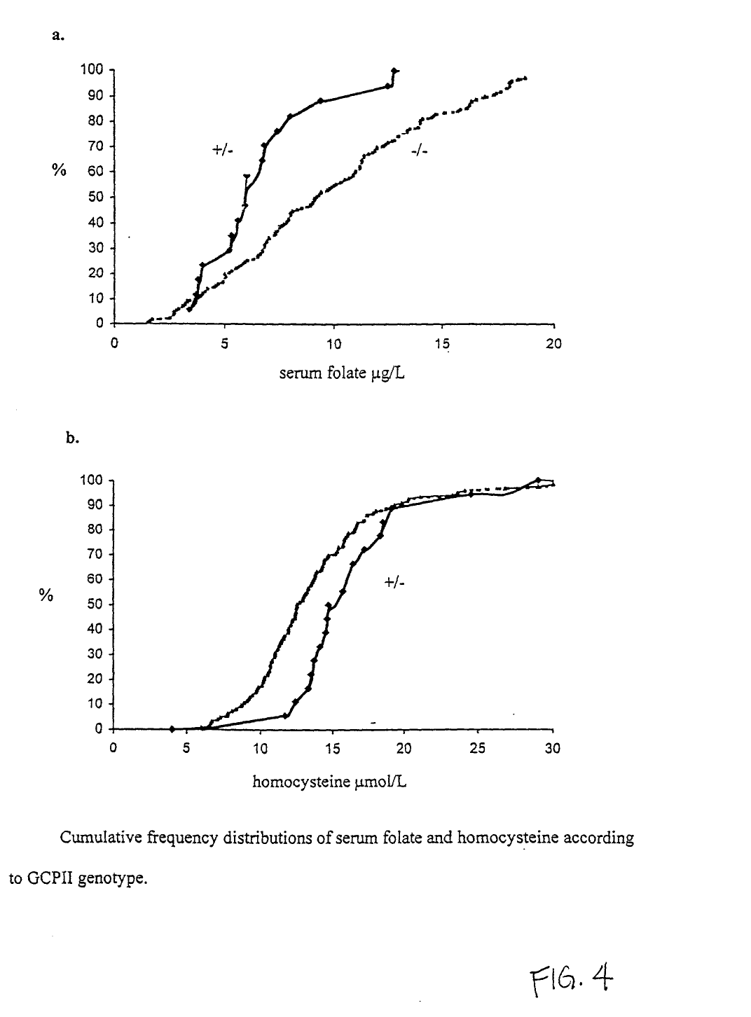

Among the subset of DNA samples from the previous study (Clarke, R. et al., Arch. Neurol. 55:1449-1455 (1998)), there were 76 samples from patients with clinically and histologically confirmed Alzheimer's Disease, 75 age-matched controls and 30 patients with dementia of unknown etiology (other). Among the study subjects, Alzheimer's Disease patients had significantly lower serum folate, RBC folate and vitamin B12 levels and significantly higher homocysteine levels than either control or other groups (p<0.0001, for each variable), confirming that the subset of DNA samples used in the present study was representative of the previous study (Clarke, R. et al., Arch. Neurol. 55:1449-1455 (1998)). Analysis by chi-squared test determined no significant segregation of the H475Y mutant allele according to diagnosis (p=0.14). Since the relationship of the H475Y GCPII allele to the disease diagnosis was not statistically significant given the sample size, all groups were consolidated for the remaining statistical analyses. Serum folate levels were significantly lower (p<0.001) and serum homocysteine levels were significantly higher (p<0.01) in H475Y positive subjects than in subjects carrying wild-type GCPII (Table 1). There was no relationship between the presence of the C677T MTHFR allele and these findings. There was no significant effect of the H475Y GCPII allele on RBC folate and vitamin B12 levels, whereas RBC folate levels were significantly associated (p<0.01) with vitamin B12 levels. Homocysteine values can be influenced by gender, age, vitamin B12, folate and creatinine levels (Nygard, O. et al., Am. J. Clin. Nutr. 67:263-270 (1998)); Bostom, A. G. et al., Kidney Int. 52:10-20 (1997)); Nygard, O. et al., JAMA 274:1526-1533 (1995)). Further analysis using two-way analysis of co-variance to control for gender, age, serum vitamin B12 and serum creatinine found a consistently significant positive (p<0.05) association of the H475Y GCPII allele with elevated homocysteine levels. Serum folate was the only confounding variable to exert a significant effect on the relationship of to with the presence of the H475Y GCPII allele (p<0.0001). This finding suggests that the significant association of higher homocysteine with the H475Y allele is a consequence of the affect of serum folate on homocysteine levels. Relationships of the presence of the H475Y GCPII allele to cumulative serum folate and homocysteine values are shown in FIG. 4.

TABLE 1
Distribution of folate, vitamin B₁₂, homocysteine and creatinine
according to GCPII genotype.

| | | GCPII genotype | |
|---|---|---|---|
| clinical parameter | all samples | +/− | −/− |
| serum folate | 9.5 ± 0.4 | 6.6 ± 0.7 | 9.8 ± 0.4* |
| (μg/L) | (n = 177) | (n = 17) | (n = 160) |
| RBC folate | 443 ± 14.0 | 379 ± 46.5 | 449 ± 14.8 |
| (μg/L) | (n = 171) | (n = 16) | (n = 155) |
| vitamin B₁₂ | 390 ± 14.7 | 324 ± 27.5 | 396 ± 15.7 |
| (ng/L) | (n = 177) | (n = 16) | (n = 161) |
| homocysteine | 14.3 ± 0.50 | 16.5 ± 1.02 | 14.0 ± 0.54† |
| (μmol/L) | (n = 141) | (n = 18) | (n = 123) |
| creatinine | 95.4 ± 1.71 | 97.0 ± 4.37 | 95.2 ± 1.83 |
| (μmol/L) | (n = 177) | (n = 17) | (n = 160) |

“+/−” means heterozygotes and “−/−” means wild-type.
Means ± SEM;
*p < 0.001,
†p < 0.01, comparing values according to genotype.

### Example 4

Analysis of Results

We characterized the cDNA sequence of human intestinal GCPII and found complete identity to human PSMA (Israeli, R. S. et al., Cancer Res 53:227-230 (1993)) and NAALADase (Luthi-Carter, R. et al., J. Pharm. Exper. Therap. 286:1020-1025 (1998)) with some exceptions. This study confirms the concept that GCPII is expressed as three different proteins: intestinal FGCP, brain NAALADase (Carter, R. E. et al., Proc. Natl. Acad. Sci. 95:3215-3220 (1998); Luthi-Carter, R. et al., J. Pharm. Exper. Therap. 286:1020-1025 (1998)), and PSMA (Israeli, R. S. et al., Cancer Res 53:227-230 (1993)). The transcriptional start site for GCPII in human jejunum was determined by RACE at nucleotide −138 relative to the translation initiation codon. This finding contrasts with the multiple transcriptional start sites determined for PSMA in prostate cancer cells at nucleotides −262, −235, and −195 relative to the translation initiation codon (Israeli, R. S. et al., Cancer Res 53:227-230 (1993)); O'Keefe, D. S. et al., Biochim. Biophys. Acta 1443:113-127 (1998)). The differences in start sites between intestine and prostate suggest that transcriptional regulation of GCPII is governed in a tissue-specific fashion. The present results show also that the 5′untranslated region of intestinal GCPII mRNA is shorter than that determined for PSMA mRNA (Israeli, R. S. et al., Cancer Res 53:227-230 (1993)); O'Keefe, D. S. et al., Biochim. Biophys. Acta 1443:113-127 (1998)). A PSMA splice variant, PSM′, was identified in human prostate cancer LNCaP cells and characterized as lacking 266 bp of the 5′region of PSMA cDNA corresponding to 146 bp of the 5′UTR and 120 bp of the ORF (Su, S. L. et al., Cancer Res. 55:1441-1443 (1995)); Grauer, L. S. et al., Cancer Res. 58:4787-4789 (1998)). PSM′ was predicted to express a cytosolic form of PSMA because the deleted sequence codes for the transmembrane domain. However, it is unlikely that PSM′ is present in the intestine since the 5′UTR region present in PSM′ is not found in intestinal GCPII mRNA. On the other hand, an intracellular enzyme capable of hydrolyzing both internal and terminal γ-glutamate residues of folylpoly-γ-glutamates was previously purified from human intestinal mucosa (Wang, T. T. Y. et al., J. Biol. Chem. 261:13551-13555 (1986)) and is encoded by a separate gene (Yao, R. et al., Proc. Natl. Acad. Sci. 93:10134-10138 (1996)). We have also identified a splice variant lacking exon 18 in human jejunum. The ratio of splice variant to wild-type GCPII transcripts could be an important underlying factor governing the level of expression of intestinal FGCP activity and potentially, brain NAALADase activity.

Further studies of human GCPII expression using Northern analysis showed its expression as a predominant 2.8 kb band in prostate, small intestine, brain, liver, kidney and spleen, consistent with previously reported findings of PSMA expression in prostate cancer cells (Israeli, R. S. et al., Cancer Res 53:227-230 (1993)) and NAALADase expression in brain (Carter, R. E. et al., Proc. Natl. Acad. Sci. 95:3215-3220 (1998)); Luthi-Carter, R. et al., J. Pharm. Exper. Therap. 286:1020-1025 (1998)). We also identified less predominant GCPII bands at 1.5 kb in the kidney and 1.0 kb in the placenta, which have not been reported previously for PSMA or NAALADase.

Using DNA samples from a previous study of aging Caucasian subjects (Clarke, R. et al., Arch. Neurol. 55:1449-1455 (1998)), we identified a C→T missense mutation predicted to replace a histidine with tyrosine at codon 475 in exon 13 of the catalytic region of GCPII. The functional significance of the missense mutation was shown by findings in vitro that the H475Y GCPII cDNA expressed less than 50% of wild-type FGCP activity in membranes of transfected COS-7 cells. Intestinal FGCP cleaves glutamate residues from folylpoly-γ-glutamates and thus plays an important regulatory role in the intestinal absorption of dietary folylpoly-γ-glutamates. Therefore, mutations in GCPII affecting the activity of FGCP would predictably decrease the intestinal absorption of dietary folylpoly-γ-glutamates and consequently decrease folate levels in body. To test this hypothesis, we analyzed DNA samples from a subset of Alzheimer's Disease patients and controls from the original English study (Clarke, R. et al., Arch. Neurol. 55:1449-1455 (1998)). Since we found no differences in the H475Y GCPII allele between Alzheimer's Disease patients and controls given the sample size available, all were combined to determine the relationship of the H475Y GCPII allele to folate and homocysteine levels. Our hypothesis was corroborated by the present findings of significantly lower (p<0.001) serum folate levels in subjects with the H475Y GCPII allele than in subjects with wild-type GCPII alleles.

Serum folate levels could also be affected by differences in dietary or supplemental intakes of folates among the experimental subjects, which was not determined in the original study (Clarke, R. et al., Arch. Neurol. 55:1449-1455 (1998)). That we observed no differences in RBC folate levels according to GCPII genotype may be explained by the confounding effect of vitamin B₁₂ levels, which were unrelated to GCPII genotype but significantly (p<0.001) affected RBC folate levels. Methylated monoglutamyl folates must be demethylated and polyglutamylated by folylpoly-γ-glutamate synthase for intracellular storage as polyglutamyl folates (Selhub, J. & Rosenberg, I. H. “Folic Acid” In Present Knowledge of Nutrition, Ziegler, E. E. & Filer, L. J. (eds.), ILSI Press, Washington, D.C., 206-219 (1996)). The demethylation reaction is catalyzed by vitamin B₁₂ and therefore, the ability of RBC to store polyglutamylated folates is dependent on vitamin B₁₂.

Serum homocysteine levels are influenced by nutritional deficiencies of folate, vitamin B₁₂, or vitamin B₆, genetic defects in one or more of the proteins involved in the metabolism of homocysteine, renal function, age and gender (Nygard, O. et al., Am. J. Clin. Nutr. 67:263-270 (1998); Bostom, A. G. et al., Kidney Int. 52:10-20 (1997); Nygard, O. et al., JAMA 274:1526-1533 (1995)); Frosst, P. et al., Nat. Genet. 10:111-113 (1995)). The thermolabile C677T mutation in MTHFR, an enzyme responsible for the synthesis of methyl tetrahydrofolate, which is required for the conversion of homocysteine to methionine has been implicated in the pathogenesis of hyperhomocysteinemia (Frosst, P. et al., Nat. Genet. 10:111-113 (1995)). In our study population, the presence of the C677T MTHFR allele had no effect on the relationship of the H475Y allele and homocysteine (Clarke, R. et al., Arch. Neurol. 55:1449-1455 (1998)). Hyperhomocysteinemia has previously been shown to be associated with Alzheimer's Disease (Joosten, E. et al., J. Gerontology 52:M76-79 (1997); Nilsson, K. et al., Eur. J. Clin. Invest. 126:853-859 (1996); Clarke, R. et al., Arch. Neurol. 55:1449-1455 (1998); McCaddon, S. et al., Int. J. Geriatr. Psychiatry 13:235-239 (1998) 14. Halsted, C. H. et al., J. Biol. Chem. 273:20417-20424 (1998)). On the other hand, in the present subset of the English study (Clarke, R. et al., Arch. Neurol. 55:1449-1455 (1998)) used for the present study we found a significant association p<0.01) of the H475Y GCPII allele with higher homocysteine levels regardless of Alzheimer's Disease status. Furthermore, controlling for the confounding variables of vitamin B₁₂, age, creatinine and gender, homocysteine levels remained significantly higher in subjects with the H475Y GCPII allele. Serum folate was the only confounder observed in this study to influence the relationship between GCPII genotype and homocysteine. This suggests that the relationship of the H475Y GCPII allele to homocysteine is secondary to the effect of the mutant allele on the serum folate level.

### Example 5

Methods and Materials

Cell Lines.

COS-7 cells (ATCC) were maintained in Dulbecco's Modified Essential Media (D-MEM) supplemented with 10% fetal calf serum, 50 units/ml penicillin G, and 50 μg/ml streptomycin. The cells were grown in at 37° C. in a 5% CO₂ incubator.

Cloning of Human Intestinal FGCP cDNA.

Two separate human jejunal cDNA libraries in pcDNA2 (Invitrogen, gift of B. Lonnerdal, University of California, Davis) and λgt10 (Clontech, gift of H. Said, University of California, Irvine) were screened with a [α³²P] dCTP labeled 852 bp PCR fragment of the ORF of the pig jejunal FGCP cDNA (Halsted, C. H. et al., J. Biol. Chem. 273:20417-20424(1998)). Standard protocols were followed for library screening (Sambrook, J. et al., Molecular Cloning, Cold Spring Harbor Press, New York (1989)). Two positive clones were identified and sequenced using the LI-COR 4200 automated sequencer (LI-COR, Lincoln, Neb.). The transcriptional start site was determined by 5′RACE using the Marathon Ready human small intestine cDNA and the Marathon RACE kit (Clontech). The first PCR reaction used the gene-specific primer: 5′-GTGCTAGCTCAACAGAATCCAGGC-3′ followed by a second PCR reaction with the nested gene-specific primer: 5′-CCACAGCCGAGTCGGT TTCGTGAA-3′. Comparative analysis with the GenBank nucleic acid database using BLASTN (Altschul, S. F. et al., Nucleic Acid Res. 25:3389-3402 (1997)) revealed that clones pFGCP52 and pFGCP72 were completely homologous to the human PSMA cDNA sequence (Israeli, R. S. et al., Cancer Res 53:227-230 (1993)) except for a 93 bp deletion in the 3′ end of clone pFGCP52. Further sequence analysis with PSMA genomic sequence (O'Keefe, D. S. et al., Biochim. Biophys. Acta 1443:113-127 (1998)) revealed the 93 bp deletion represented exon 18. The presence of the splice variant in human intestine was confirmed by RT-PCR of human jejunal mucosal RNA. A specimen of normal adult human jejunum was obtained at surgery from an obese patient undergoing elective gastric bypass with construction of gastro-jejunal anastomosis. The jejunal mucosa was scraped with a glass slide then immediately placed in TRIzol reagent (Life Technologies), frozen in liquid nitrogen and stored at −80° C. until RNA extraction.

Total RNA was extracted using TRIzol reagent followed by reverse transcription using Superscript reverse transcriptase (Life Technologies) and PCR of the first strand cDNA using primers flanking exon 18, forward in exon 16: 5′-GAAACAA ACAAATTCAGCGG-3′ and reverse in exon 19: 5′-AGAGCATCATAAATTCCTGG-3′. BLASTP (Altschul, S. F. et al., Nucleic Acid Res. 25:3389-3402 (1997)) was used to compare the predicted amino acid sequence of human intestinal GCPII cDNA with the Genbank protein database.

Northern Analysis.

Selected human multiple tissue northern blots (Clontech) were hybridized at 42° C. for 18 hrs with clone pFGCP52 labeled with [α³²P]dCTP using the RadPrime DNA Labeling system (Life Technologies). Following hybridization and washing, the blots were exposed to X-OMAT AR film (Kodak, Rochester, N.Y.) for 48 hrs with intensifying screens at −80° C.

Human GCPII Mutation Analysis.

Genomic DNA samples were obtained from a prior published study of Caucasian subjects including 76 samples from patients with Alzheimer's Disease, 30 samples from patients with dementia of unknown etiology and 75 samples from their age-matched controls (Clarke, R. et al., Arch. Neurol. 55:1449-1455 (1998)). Exon and exon-intron borders of GCPII were amplified in each DNA sample using the following primers designed from the reported genomic PSMA sequence (O'Keefe, D. S. et al., Biochim. Biophys. Acta 1443:113-127 (1998)): exon 2,5′-GTATCTCAT GCCTATTTTTGG-3′ and 5′-GTCCATATAAACTTTCGAGGA-3′; exon 3,5′-CCAC TTTCAATTTTTGTTCACC-3′,5′-ACATCATAATGAGCTAGCTCAA-3′, 5′-CCTGT TGTCCTACCCAAA-3′ and 5′-AAATGGGGGGAATGTTTC-3′; exon 4,5′-GTAAT CAAACTTTAACTG-3′ and 5′-GTCATTTTTATTTGTTGC-3′; exon 5,5′-AAGTCA TTCCCGATCTCAA-3′ and 5′-AACTTTTTACATTGGGGGA-3′; exon 6,5′-AAAG ACACTGTGTTTTCTAG-3′ and 5′-AAGTTTCCTATGATATTCAA-3′; exon 7, 5′-A ACTGAGTTTTTACTGGAAA-3′ and 5′-ACTGTCAATAAGAAATTAC-3′; exon 8,5′-AACATTTTAATGCGTTACTTCAA-3′ and 5′-CCCCTTAAA AGAGTTAAAATTA A -3′; exon 9,5′-AATTCCCTTAAACCTTATAG-3′ and 5′-ACTTGATCAATATTTG CTAA-3′; exon 10,5′-AATCTTGTATCTATGCTTTCTAG-3′ and 5′-AGAAGACTA AACTGAGACTCAG-3′; exon 11,5′-GGGCTTGGTAGTGTCCTGG-3′ and 5′-GC TTGGCAAACAAGTCCTGG-3′; exon 12, 5′-AATCTAC ATACATTAAA TGTAG-3′ and 5′-AGTGCATAATTTATTAACTAGA-3′; exon 13,5′-CATTCTGGTAGGAATT TAGCA-3′ and 5′-AAACACCACCTATGTTTAACA-3′; exon 14,5′-AATGGACATC TAGTTTTTAA-3′ and 5′-ACTTAATGATTGAAAGAAAA-3′; exon 15,5′-AAGAAT GGGGTTTAGTTTAA-3′ and 5′-AGTCAGAATTATTTGTTTAG-3′; exon 16,5′-AA TAGTAAATATGGTGCTAA-3′,5′-ACAGTGAGGTGATATTTAAA-3′,5′-AAATATC ACCTCACTGTGG-3′ and 5′-AATAGAACCATACAGATGAG-3′; exon 17,5′-AAG CATTTGATGATCCCAA-3′ and 5′-AAAGCTAGTTCAGCAACAG-3′; exon 18,5′-GGTAGAAACATATTTTCATGAA-3′ and 5′-AAAAAGTGATATTACAGAAAGG-3′; exon 19,5′-GGGCACCTAATAAACAGCAAA-3′ and 5′-GGCTACTTCACTCAAA GTCTC-3′. Each PCR product was screened for polymorphisms by temperature modulated heteroduplex chromatography (Kuklin, A. et al., Genetic Testing 1:201-206 (1997/1998)) using the WAVE DNA Fragment Analysis System (Transgenomic Inc, San Jose, Calif.). PCR samples found to contain polymorphisms and representative wild-type samples were purified using the QIAquick PCR purification kit (QIAGEN) and both strands were sequenced using the PCR primers and an ABI Prism 377 DNA automated sequencer.

Transfection Studies.

Monkey kidney COS-7 cells were transfected with DNA constructs of the wild-type GCP II cDNA or GCPII cDNA containing the H475Y missense mutation. The wild-type GCPII cDNA was synthesized by PCR of reverse transcribed human jejunal mucosal RNA using Pfu polymerase (Stratagene) and primers at the 5′ end based on the RACE sequence and the 3′ end based on the clone pFGCP52 sequence. The wild-type GCPII cDNA was cloned into the mammalian expression vector, pTRACER-CMV2 (Invitrogen). The H475Y missense mutation was introduced into GCPII cDNA in pTRACER-CMV2 using the QuickChange Site-Directed Mutagenesis Kit (Stratagene). The primers used in the PCR reaction were: 5′-CCGCTGATGTACAGCTTGGTATAC AACCTAACAAAAG-3′ and 5′-CTTTTGTTAGGTGTATACCAAGCTGTACATCAG CGG-3′. The resulting product was transformed into XL1-Blue cells and the mutated sequence confirmed by sequencing using an ABI Prism 377 DNA automated sequencer. Transfections of COS-7 cells with wild-type and the H475Y mutant forms of GCPII were accomplished using LipofectAMINE and OPTI-MEM I medium (Life Technologies) according to the manufacturer's protocol. One day prior to transfection, the cells were seeded onto 6-well plates and grown in serum-containing DMEM so that on the day of transfection the cells were 60-80% confluent. Cells membranes were harvested at 48 hrs post-transfection by scraping into 50 mM tris-HCl (pH 7.4) followed by homogenization and removal of supernatant after centrifugation (35,000×g) for 30 min. The membrane pellets were resuspended in 50 mM tris-HCl and the protein concentration of the membranes was determined using the enhanced protocol BCA assay (Bio-Rad). FGCP activity was determined in COS-7 transfectant membranes using folyl-γ-glutaryl-↓-[¹⁴C]glutamate and a modification (Chandler, C. J. et al., J. Biol. Chem. 261:928-933 (1986)) of the method of Krumdieck and Baugh (Krumdieck, C. L. et al., Anal. Biochem. 35:123-129 (1970)). Expression of green fluorescence protein was used to monitor transfection efficiency and uniformity. Mock transfected cells served as controls.

Statistical Analyses.

The Pearson's Chi-Squared test was used to determine the relationship between the H475Y GCPII allele and Alzheimer's Disease diagnosis. One-way analysis of variance was used to determine the relationship between Alzheimer's Disease diagnosis and serum folate, RBC folate, vitamin B₁₂, homocysteine, and creatinine. Two-way analysis of variance was used to determine if the presence of the H475Y GCPII allele and Alzheimer's Disease diagnosis had an additive effect on serum folate, RBC folate, vitamin B₁₂ and homocysteine. Since no significant interaction was found the Student's t-test was used to determine the significance of differences in serum folate, RBC folate, vitamin B₁₂ and homocysteine between mutation positive and negative subjects. Two-way analysis of co-variance was used to further analyze the association of the H475Y GCPII allele with homocysteine levels, controlling for the confounding variables of gender, age, vitamin B₁₂ and creatinine.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening an individual for increased risk of low folate status, said method comprising detecting a mutation in a human glutamate carboxypepidase II (GCPII) gene in a biological sample from said individual, wherein detection of the mutation is indicative of decreased ability to hydrolyse a terminal glutamate residue of a folypoly-γ-glutamate, which decreased ability is associated with low folate status.

2. The method of claim 1, wherein the mutation is a single nucleotide polymorphism.

3. The method of claim 3, wherein the single nucleotide polymorphism causes an amino acid substitution of H475Y.

4. A method of claim 1 wherein the mutation is detected by
(a) amplifying the GCPII gene, or a portion thereof containing the mutation, with a set of primers to provide an amplified product,
(b) sequencing the amplified product to obtain a sequence, and
(c) comparing the sequence of the amplified product with a known sequence of a wild-type GCPII gene,
wherein a difference between the sequence of the amplified product and the sequence of the wild-type GCPII gene indicates the presence of a mutation.

5. A method of claim 4, wherein said amplification is by polymerase chain reaction.

6. A method of claim 4, wherein said sequencing is performed by detecting the incorporation of a nucleotide into a strand complementary to a template strand by detecting the presence of a pyrophosphate released from the incorporated nucleotide.

7. A method of claim 1 wherein the mutation is detected by
(a) amplifying exon 13 of the GCPII gene with a set of primers to provide an amplified product,
(b) sequencing the amplified product to obtain a sequence, and
(c) comparing the sequence of the amplified product with a known sequence of exon 13 of a wild-type GCPII gene,
wherein a difference between the sequence of the amplified product and the sequence of the wild-type GCPII gene indicates the presence of a mutation.

8. A method of claim 7, wherein said primers are 5′-CATTCTGGTAGGAATT TAGCA-3′ and 5′-AAACACCACCTATGTTTAACA-3′.

9. A method of claim 7, wherein said amplification is by polymerase chain reaction.

10. A method of claim 7, wherein said sequencing is performed by detecting the incorporation of a nucleotide into a strand complementary to a template strand by detecting the presence of a pyrophosphate released from the incorporated nucleotide.

11. A method of claim 1, wherein said mutation is detected by hybridizing DNA from said individual to a test nucleic acid under stringent conditions.

12. A method of claim 11, wherein either said DNA from said individual or said test nucleic acid is immobilized on a solid support.

13. A method of claim 1, wherein said mutation is detected by
(a) amplifying exon 13 said GCPII gene,
(b) subjecting said amplified exon 13 to digestion by restriction enzymes,
(c) separating the resulting restriction products to form a pattern of restriction fragment lengths, and
(d) comparing the pattern of restriction fragment lengths to a pattern of restriction fragment lengths formed by subjecting amplified exon 13 of a wild-type GCPII gene to the same restriction enzymes.

14. A method of claim 13, wherein said separation of the restriction products is by gel electrophoresis.

15. A method of claim 13, wherein the restriction enzyme is AccI.

16. A method of claim 15, wherein the pattern of restriction fragments of exon 13 of the GCPII gene of the individual shows restriction fragments selected from the group consisting of: 141 bases and 103 bases.

17. A method of claim 1, wherein said mutation is detected by specifically binding an antibody to a truncated product of the GCPII gene, wherein the specific binding of the antibody to the truncated gene product is indicative of a mutation impairing the ability of the GCPII gene product to digest a dietary folate.

18. A method of claim 17, wherein detection of said specific binding of said antibody and said truncated gene product is by ELISA.

19. A method of screening an individual for increased risk of low folate status comprising
(a) performing reverse transcriptase-PCR on mRNA from intestinal cells of the individual to amplify products of a GCPII gene, and
(b) determining the ratio of a variant product in which 93 bases of exon 18 are deleted to a normal product of the GCPII gene,
wherein a ratio of the variant form to the normal form greater than 1:3 indicates the individual is at increased risk of low folate status.

20. A mutation in a GCPII gene which impairs the ability of a product of the gene to hydrolyse a conjugated folate to release folic acid compared to a product of a wild-type GCPII gene.

21. A mutation of claim 20, wherein the ability of a product of the gene to hydrolyse a conjugated folate is reduced by 20 percent or more compared to a product of a wild-type GCPII gene.

22. A mutation of claim 20, wherein the mutation is a 93-base deletion resulting from the elimination of exon 18.

23. The mutation of claim 20, wherein the mutation is a single nucleotide polymorphism.

24. The mutation of claim 23, wherein the single nucleotide polymorphism causes an amino acid substitution of: H475Y.

25. A kit for the detection of a woman at increased risk for bearing a child with a neural tube defect, comprising:
(a) a container, and
(b) primers for amplifying a GCPII gene or portion thereof.

26. A kit of claim 25, further comprising instructions for detecting a mutation in the GCPII gene resulting in decreased ability of a product of the GCPII gene to hydrolyze a conjugated folate compared to the product of a wild-type GCPII gene.

27. A kit of claim 25, further comprising an AccI restriction enzyme.

28. A kit for the detection of an individual at increased risk for low folate status, comprising:
(a) a container, and
(b) primers for amplifying a GCPII gene or portion thereof.

29. A kit of claim 28, further comprising instructions for detecting a mutation in the GCPII gene resulting in decreased ability of a product of the GCPII gene to hydrolyze a conjugated folate compared to a product of a wild-type GCPII, wherein detection of such a mutation indicates the individual is at increased risk for low folate status.

30. A kit of claim 28, further comprising an AccI restriction enzyme.
